(19) United States
(12) Patent Application Publication
(10) Pub. No.: US 2023/0104559 A1
(43) Pub. Date: Apr. 6, 2023

(54) BONE FUSION PLATE FOR TREATING THE HALLUX

(71) Applicant: CONMED Corporation, Largo, FL (US)

(72) Inventors: Bradley Aaron Spencer Cowan, Memphis, TN (US); Alan G. Taylor, Memphis, TN (US); Rebecca Hawkins Wahl, Escondido, CA (US)

(73) Assignee: CONMED Corporation, Largo, FL (US)

(21) Appl. No.: 17/959,782

(22) Filed: Oct. 4, 2022

Related U.S. Application Data
(60) Provisional application No. 63/251,960, filed on Oct. 4, 2021.

(51) Int. Cl. A61B 17/80

(52) U.S. Cl. CPC A61B 17/8061; A61B 17/8057; A61B 2017/565

(57) ABSTRACT

An apparatus and methods are provided for a bone fusion plate for treating deformities of the hallux. The bone fusion plate includes a width and a length and screw hole position and trajectory suitable for fixating a 1^{st} metatarsal bone and a metatarsal head during a straight distal osteotomy to correct a hallux valgus deformity of a patient. The bone fusion plate includes a plate portion comprising a lower, distal surface and a buttress comprising a proximal surface. The distal surface fastens onto the metatarsal head while the proximal surface fastens onto a proximal bone surface exposed by cutting the 1^{st} metatarsal bone and laterally shifting the metatarsal head during the straight distal osteotomy. Threaded countersunk screw holes in the buttress and the plate portion receive bone screws for fastening onto the 1^{st} metatarsal bone and the metatarsal head. The countersinks prevent the bone screws from irritating surrounding soft tissues.

### PRIORITY

This application claims the benefit of and priority to U.S. Provisional Application, entitled “Bone Fusion Plate For Treating The Hallux,” filed on Oct. 4, 2021, and having application Ser. No. 63/251,960, the entirety of said application being incorporated herein by reference.

### FIELD

Embodiments of the present disclosure generally relate to securing bones together. More specifically, embodiments of the disclosure relate to an apparatus and methods for a bone fusion plate for a lateral head shift osteotomy of a 1^{st} metatarsal bone for corrective procedures of the hallux.

### BACKGROUND

Hallux valgus is a progressive foot deformity wherein the distal region of the big toe (i.e., the “hallux”) deviates in a lateral direction. Such a deformity can be caused by wearing pointed shoes with a narrow toe box. For example, when wearing high heel shoes, the foot is forced into the front of the shoe. The narrow front of the shoe forces the distal hallux in the lateral direction, toward the other toes, while a distal portion of the 1^{st} metatarsal head is forced in a medial direction. Forcing the distal metatarsal head in the medial direction pushes it outward and against an edge of the shoe. The irritation caused by pressing the metatarsal head against the shoe often causes an enlarged and thickened callus, or a bunion, to form.

A hallux valgus deformity may have significant ramifications for soft tissue problems in other areas, such as pain and functional deficit. For example, a hallux valgus deformity can give rise to an impaired gait characterized by lateral and posterior weight shift, late heel rise, decreased single-limb balance, pronation deformity, and the like. When the hallux is deviating away from its normal position, it does not have the mechanical ability to perform these tasks correctly. For example, if the hallux is not preventing overpronation, a number of other problems may develop, including plantar fasciitis, shin splints, or other ankle or knee pathologies.

Given that hallux valgus is relatively prevalent in the general population, there is an ongoing need for the development of foot treatment capabilities such as that related to, for example, treating hallux valgus deformities. Provided herein are embodiments and methods for a bone fusion plate for a lateral head shift osteotomy of a 1^{st} metatarsal bone for corrective procedures of the hallux.

### SUMMARY

An apparatus and methods are provided for a bone fusion plate and screw construct for treating deformities of the hallux. The bone fusion plate includes a width and length and screw hole position and trajectory suitable for fixating a 1^{st} metatarsal bone shaft and a metatarsal head after a straight distal lateral slide osteotomy to correct a hallux valgus deformity of a patient. Similarly, a Tailor's bunion in the 5^{th} metatarsal could be treated with a medial slide osteotomy and fixated with this buttress plate and screws. The plate also assists as a cutting guide for positioning and performing the osteotomy. The bone fusion plate includes a plate portion comprising a lower, distal surface and a buttress comprising a proximal surface. The distal surface is adapted to be attached to a side surface of a metatarsal head while the proximal surface is configured to be attached to a proximal bone surface formed by cutting the 1^{st} metatarsal bone and laterally shifting the metatarsal head. The distal surface and the proximal surface share an intervening buttress angle suitable for fixating the 1^{st} metatarsal bone and the metatarsal head. Threaded countersunk screw holes in the plate portion and the buttress receive bone screws for attaching to the 1^{st} metatarsal bone and the metatarsal head. The countersunk holes cause the bone screws to reside below an upper surface of the plate portion to avoid irritating surrounding soft tissues.

In an exemplary embodiment, a bone fusion plate for treating deformities of the hallux comprises: a plate portion comprising a lower, distal surface; and a buttress comprising a proximal surface.

In another exemplary embodiment, the bone fusion plate includes a width and a length suitable for fixating a 1^{st} metatarsal bone and a metatarsal head. In another exemplary embodiment, the plate portion includes a thickness and the buttress includes an overall height that is about 4 mm greater than the thickness.

In another exemplary embodiment, the buttress includes one or more proximal screw holes configured to receive bone screws for attaching the buttress to a proximal bone surface of a 1^{st} metatarsal bone. In another exemplary embodiment, any one or more of the one or more proximal screw holes includes a variable angle feature, which allows screws to be placed off-axis, disposed on a proximal backside of the buttress. In another exemplary embodiment, the buttress includes a channel configured to receive a K-wire for provisionally positioning the buttress with respect to the 1^{st} metatarsal bone. In another exemplary embodiment, any one or more of the one or more proximal screw holes includes a threaded countersink for threadably engaging a head portion of a proximal bone screw. In another exemplary embodiment, the threaded countersink is configured to cause the head portion to reside below an exterior surface of the buttress so as to avoid irritating surrounding soft tissues.

In another exemplary embodiment, the one or more proximal screw holes are disposed in the buttress at a proximal screw angle with respect to an upper surface of the plate portion. In another exemplary embodiment, the proximal screw angle is adapted to direct the bones screws into a lateral cortex of the 1^{st} metatarsal bone. In another exemplary embodiment, any one or more of the one or more proximal screw holes includes a conical relief disposed on a proximal backside of the buttress. In another exemplary embodiment, the conical relief is adapted to allow for variable screw angulation for a range of osteotomy shifts.

In another exemplary embodiment, the plate portion includes one or more distal screw holes configured to receive bone screws for attaching the plate portion to a metatarsal head. In another exemplary embodiment, any one or more of the one or more distal screw holes are positioned in an offset arrangement. In another exemplary embodiment, the offset distal screw holes are incorporated into the offset bone fusion plate in a right-specific configuration or a left-specific configuration. In another exemplary embodiment, the plate portion includes a pin hole configured to receive a K-wire or an olive wire for provisionally attaching the plate portion to the metatarsal bone. In another exemplary embodiment, any one or more of the one or more distal screw holes includes a threaded countersink for threadably engaging a head portion of a distal bone screw. In another exemplary embodiment, the threaded countersink is configured to cause the head portion to reside below an upper surface of the plate portion to avoid irritating surrounding soft tissues.

In another exemplary embodiment, the distal surface is adapted to be attached to a side surface of a metatarsal head. In another exemplary embodiment, the proximal surface is configured to be attached to a proximal bone surface formed by cutting a 1^{st} metatarsal bone and laterally shifting the metatarsal head. In another exemplary embodiment, the distal surface and the proximal surface share an intervening buttress angle suitable for fixating the 1^{st} metatarsal bone and the metatarsal head. In another exemplary embodiment, the buttress angle is configured to guide cutting the 1^{st} metatarsal bone. In another exemplary embodiment, the buttress angle ranges between about 70 degrees and 110 degrees.

In an exemplary embodiment, a bone fusion plate for treating hallux valgus comprises: a plate portion comprising a lower, distal surface; and a buttress comprising a proximal surface.

In another exemplary embodiment, the buttress includes one or more proximal screw holes configured to receive bone screws for attaching the buttress to a proximal bone surface of a metatarsal bone. In another exemplary embodiment, any one or more of the one or more proximal screw holes includes a conical relief configured to allow for variable screw angulation. In another exemplary embodiment, any one or more of the one or more proximal screw holes includes a threaded countersink for threadably engaging a head portion of a proximal bone screw. In another exemplary embodiment, the one or more proximal screw holes are disposed in the buttress at a proximal screw angle with respect to the plate portion. In another exemplary embodiment, any one or more of the one or more proximal screw holes includes a conical relief adapted to allow for variable screw angulation for a range of osteotomy shifts.

In another exemplary embodiment, the plate portion includes one or more distal screw holes configured to receive bone screws for attaching the plate portion to a metatarsal head. In another exemplary embodiment, the one or more distal screw holes are positioned at different distances from the buttress. In another exemplary embodiment, any one or more of the one or more distal screw holes includes a threaded countersink for threadably engaging a head portion of a distal bone screw.

In another exemplary embodiment, the proximal surface is configured to be attached to a proximal bone surface formed by cutting a metatarsal bone and laterally shifting the metatarsal head. In another exemplary embodiment, the proximal surface and the distal surface share an intervening buttress angle suitable for fixating the metatarsal bone and the metatarsal head. In another exemplary embodiment, the buttress angle comprises a cut guide configured to guide cutting the metatarsal bone. In another exemplary embodiment, the buttress angle ranges between about 70 degrees and about 110 degrees.

In an exemplary embodiment, a lateral shift osteotomy buttress plate comprises: a plate portion for attaching to a distal bone portion; and a buttress for attaching to a proximal bone portion.

In another exemplary embodiment, the buttress includes a proximal surface disposed at a buttress angle with respect to a distal surface comprising the plate portion. In another exemplary embodiment, the proximal surface comprises a cut guide for performing a bone cut to separate the distal bone portion and the proximal bone portion. In another exemplary embodiment, the buttress angle ranges between about 70 degrees and about 110 degrees.

In another exemplary embodiment, the buttress includes one or more proximal screw holes disposed at a proximal screw angle with respect to the plate portion and configured to receive bone screws for attaching the buttress to the proximal bone portion. In another exemplary embodiment, any one or more of the one or more proximal screw holes includes a threaded countersink for threadably engaging a head portion of a proximal bone screw. In another exemplary embodiment, any one or more of the one or more proximal screw holes includes a conical relief configured to allow for variable screw angulation for a range of osteotomy shifts.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary-use environment wherein a straight distal osteotomy has been performed on a 1^{st} metatarsal bone, according to the present disclosure;

FIG. 2 illustrates a ghost view of a 1^{st} metatarsal bone that has been treated with a straight distal osteotomy to correct a hallux valgus deformity in accordance with the present disclosure;

FIG. 3 illustrates a top view of an exemplary embodiment of a bone fusion plate for performing a straight distal osteotomy in accordance with the present disclosure;

FIG. 4 illustrates a side view of the bone fusion plate of FIG. 3;

FIG. 5 is a cross-sectional view of the bone fusion plate of FIG. 3, taken along a line 5-5, illustrating a conical relief angle provided to bone screws;

FIG. 6 is a cross-sectional view of the bone fusion plate of FIG. 3, taken along a line 6-6, illustrating threads disposed within a bone screw hole;

FIG. 7 illustrates an exemplary-use environment wherein a 1^{st} metatarsal bone has been treated with a bone fusion plate to correct a hallux valgus deformity in accordance with the present disclosure;

FIG. 8A illustrates an exemplary embodiment of a bone fusion plate attached to a 1^{st} metatarsal bone in preparation for performing a straight bone cut, according to the present disclosure;

FIG. 8B illustrates an exemplary-use environment wherein a metatarsal head of a 1^{st} metatarsal bone is being pushed in a lateral direction by way of a lever device;

FIG. 8C illustrates the exemplary-use environment of FIG. 8B wherein the metatarsal head is being held in a suitable position with respect to the 1^{st} metatarsal bone;

FIG. 9A illustrates a threaded handle device coupled with an exemplary embodiment of a bone fusion plate that is attached to a 1^{st} metatarsal bone in preparation for performing a straight bone cut, according to the present disclosure;

FIG. 9B illustrates the exemplary-use environment of FIG. 9A wherein the metatarsal head is being held in a suitable position with respect to the 1^{st} metatarsal bone;

FIG. 10A illustrates an exemplary-use environment wherein a lever device is cooperating with a bone fusion plate to provisionally fixate a metatarsal head with respect to a 1^{st} metatarsal bone according to the present disclosure;

FIG. 10B illustrates the exemplary-use environment of FIG. 10A wherein a K-wire is being used to fixate the metatarsal head;

FIG. 10C illustrates the exemplary-use environment of FIG. 10B wherein the K-wire and the bone fusion plate are cooperating to fixate the metatarsal head in a suitable provisional position with respect to the 1^{st} metatarsal bone, in accordance with the present disclosure;

FIG. 11 illustrates an exemplary-use environment wherein an offset bone fusion plate has been used to perform a straight distal osteotomy on a 1^{st} metatarsal bone, according to the present disclosure;

FIG. 12 illustrates a ghost view of a 1^{st} metatarsal bone that has been treated with a straight distal osteotomy, by way of an offset bone fusion plate, to correct a hallux valgus deformity in accordance with the present disclosure;

FIG. 13 illustrates an exemplary-use environment wherein a 1^{st} metatarsal bone has been treated with an angled bone fusion plate to correct a hallux valgus deformity in accordance with the present disclosure;

FIG. 14 illustrates an exemplary-use environment wherein a 1^{st} metatarsal bone has been treated with an angled bone fusion plate to correct a hallux valgus deformity in accordance with the present disclosure;

FIG. 15 illustrates an exemplary-use environment wherein a 1^{st} metatarsal bone has been treated with a straight bone fusion plate to correct a hallux valgus deformity in accordance with the present disclosure;

FIG. 16A illustrates an exemplary-use environment wherein a 1^{st} metatarsal bone has been treated with a T-shaped bone fusion plate to correct a hallux valgus deformity in accordance with the present disclosure; and

FIG. 16B illustrates an exemplary-use environment wherein a 1^{st} metatarsal bone has been treated with a T-shaped bone fusion plate to correct a hallux valgus deformity in accordance with the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the bone fusion plate, screw constructs, and methods disclosed herein may be practiced without these specific details. In other instances, specific numeric references such as “first portion,” may be made. However, the specific numeric reference should not be interpreted as a literal sequential order but rather interpreted that the “first portion” is different than a “second portion.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

A hallux valgus deformity can cause soft tissue problems, such as pain and functional deficit. For example, a hallux valgus deformity can give rise to an impaired gait characterized by lateral and posterior weight shift, late heel rise, decreased single-limb balance, pronation deformity, and the like. When the hallux is deviating away from its normal position, a number of other problems may develop, including plantar fasciitis, shin splints, or other ankle or knee pathologies. Given that hallux valgus is relatively prevalent in the general population, there is an ongoing need for the development of foot treatment capabilities such as that related to, for example, treating hallux valgus deformities. Provided herein are embodiments and methods for a bone fusion plate for fixating a 1^{st} metatarsal bone of a patient for corrective procedures of the hallux. Further, the embodiments and methods are presented herein for fixating a 5^{th} metatarsal of a patient, such as by way of a medial slide osteotomy and fixation, for correcting a Tailor's bunion.

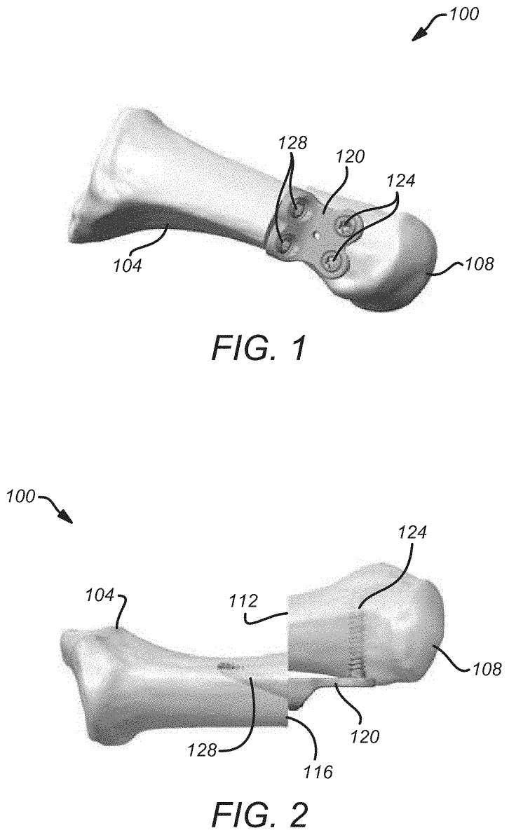

FIGS. 1-2 illustrate an exemplary-use environment wherein a straight distal osteotomy 100 has been performed on a 1^{st} metatarsal bone 104 to correct a hallux valgus deformity of a patient. As best shown in FIG. 2, a straight bone cut has been performed to separate a metatarsal head 108 from the 1^{st} metatarsal bone 104. The straight bone cut has exposed a distal bone surface 112 of the metatarsal head 108 and a proximal bone surface 116 comprising the 1^{st} metatarsal bone 104. The straight distal osteotomy 100 comprises moving the metatarsal head 108 in a lateral direction to a suitable position relative to the 1^{st} metatarsal bone 104 and then fixating the position by way of a bone fusion plate 120. The bone fusion plate 120 is attached to the metatarsal head 108 by way of distal bone screws 124 that are driven into bone holes drilled into the side of the metatarsal head 108. Further, the bone fusion plate 120 is attached to the 1^{st} metatarsal bone by way of proximal bone screws 128 that are driven into bone holes drilled into the proximal bone surface 116. As will be appreciated, the 1^{st} metatarsal bone 104 and the metatarsal head 108 will fuse together over time as bone tissue grows between distal and proximal bone surfaces 112, 116.

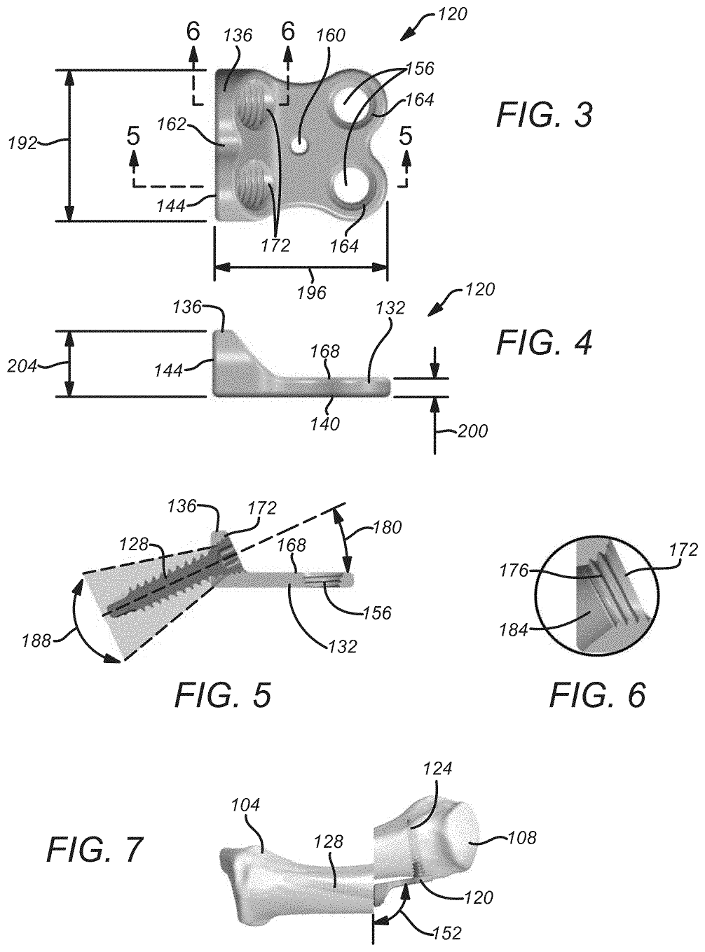

FIGS. 3-4 illustrate an exemplary embodiment of a bone fusion plate 120 configured for guiding and fixating a lateral head shift osteotomy of the 1^{st} metatarsal bone 104, as shown in FIGS. 1-2, in accordance with the present disclosure. As shown in FIGS. 3-4, the bone fusion plate 120 includes a plate portion 132 and a buttress 136. The plate portion 132 generally is a planar member having a lower, distal surface 140 that is adapted to be attached to the surface of the metatarsal head 108 (see FIG. 2). The buttress 136 generally is a thicker portion of the bone fusion plate 120 and includes a proximal surface 144 configured to be attached to the proximal bone surface 116 of the 1^{st} metatarsal bone 104 (see FIG. 2). The distal and proximal surfaces 140, 144 share an intervening buttress angle 152 (see FIG. 7) suitable for fixating the 1^{st} metatarsal bone 104 and the metatarsal head 108 as shown in FIGS. 1-2. The buttress angle 152 may be used to guide cutting the 1^{st} metatarsal bone 104, as described herein. In the embodiment of FIGS. 3-4, the buttress angle 152 is about 90 degrees. In other embodiments, however, the buttress angle 152 may range between about 70 degrees and about 110 degrees, without limitation. For example, the buttress angle 152 shown in FIG. 7 is about 100 degrees to facilitate creating a 10-degree offset cut of the neck of the metatarsal head 108 before the straight cut is performed on the 1^{st} metatarsal bone 104.

As shown in FIG. 3, the plate portion 132 includes distal screw holes 156 and a pin hole 160. The distal screw holes 156 are configured to receive distal bone screws 124 for attaching the bone fusion plate 120 to the metatarsal head 108 as shown in FIG. 2. The distal screw holes 156 may include threaded countersinks 164, as shown in FIG. 3. The threaded countersinks 164 facilitate threadably engaging a head portion of each distal bone screw 124 as well as causing the head portion to reside below an upper surface 168 of the plate portion 132 so as to avoid irritating surrounding soft tissues. The pin hole 160 is configured to enable a practitioner, such as a surgeon, to provisionally position the bone fusion plate 120 prior to attaching the plate portion 132 to the metatarsal head 108 by way of the distal bone screws 124. As such, a K-wire or an olive wire may be inserted through the pin hole 160 for provisionally attaching the plate portion 132 to the metatarsal bone 108. Further, a channel 162 disposed in the buttress 136 may be used by the surgeon to provisionally position the bone fusion plate 132 prior to attaching the buttress 136 to the 1^{st} metatarsal bone 104 by way of the proximal bone screws 128, as described herein.

With continuing reference to FIG. 3, the buttress 136 includes proximal screw holes 172 adapted to receive proximal bone screws 128 for attaching the bone fusion plate 120 to the 1^{st} metatarsal bone 104 as shown in FIG. 2. The proximal screw holes 172 preferably include threaded countersinks 176 (see FIG. 6) configured to threadably engage a head portion of each proximal bone screw 128. Further, it is contemplated that the threaded countersinks 176 cause the head portion to reside below an exterior surface of the buttress 136 so as to avoid irritating surrounding soft tissues.

Moreover, as shown in FIG. 5, the proximal screw holes 172 are disposed in the buttress 136 at a proximal screw angle 180 with respect to the upper surface 168 of the plate portion 132. The proximal screw angle 180 may comprise any suitable angular value, without limitation. In some embodiments, the proximal screw angle 180 may comprise an angle that converges or diverges with respect to the upper surface 168 of the plate portion 132. In some embodiments, the proximal screw angle 180 may be about 25 degrees so as to direct the proximal bones screws 128 into the lateral cortex of the 1^{st} metatarsal bone 104 for fixating the buttress 136 to the 1^{st} metatarsal bone 104. As shown in FIG. 6, a conical relief 184 is disposed on a proximal side of the buttress 136, behind the threaded countersinks 176. The conical relief 184 includes an overall angle 188, shown in FIG. 5, that allows the proximal bone screws 128 to be placed off-axis with respect to the proximal screw holes 172. In some embodiments, the overall angle 188 comprises about 30 degrees to allow for variable screw angulation for a range of osteotomy shifts.

Turning, again, to FIGS. 3-4, the bone fusion plate 120 includes a width 192 and a length 196 suitable for fixating the 1^{st} metatarsal bone 104 and the metatarsal head 108 as shown herein. In an embodiment, the width 192 is about 11 millimeters (mm) and the length is about 14.8 mm. In some embodiments, however, the width 192 may range between about 11 mm and about 16 mm. In some embodiments, the width 192 may range between roughly 13 mm and about 16 mm, without limitation. Further, the plate portion 132 includes a thickness 200 while the buttress 136 includes an overall height 204 that is generally 4 mm greater than the thickness 200 of the plate portion 132. In one embodiment, for example, the thickness 200 is about 1.5 mm and the height 204 is about 5.5 mm. In another embodiment, however, the thickness 200 is about 1.6 mm and the height 204 is about 5.6 mm.

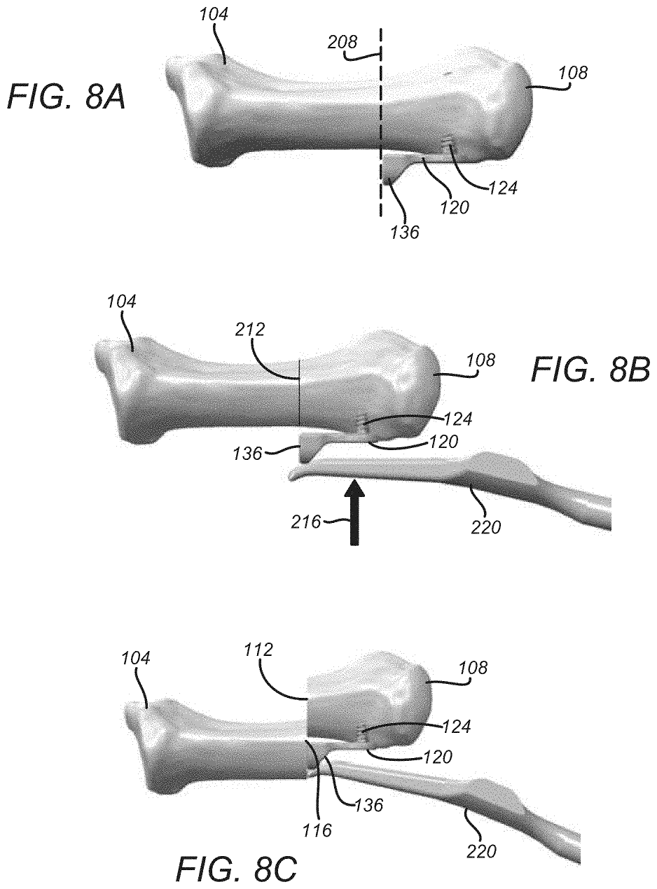

Turning, now, to FIGS. 8A through 8C, steps are illustrated for using the bone fusion plate 120 to perform a lateral head shift osteotomy of the 1^{st} metatarsal bone 104 to correct a hallux valgus deformity, or other similar condition of the hallux. As specifically shown in FIG. 8A, the bone fusion plate 120 may be fastened to the metatarsal head 108 by way of distal bone screws 124. It is contemplated that the bone fusion plate 120 may be provisionally fixed to the metatarsal head 108 by way of an olive wire or a K-wire passed through the pin hole 160. A bone drill and drill guide towers (not shown) may be used to prepare bone holes suitable to receive the distal bone screws 124. Once the bone fusion plate 120 is fastened to the metatarsal head 108, the buttress 136 may be used to indicate a straight bone cutline 208 across the 1^{st} metatarsal bone 104. It is contemplated that the buttress 136 may be used as a guide during cutting the 1^{st} metatarsal bone 104 along the straight bone cutline 208.

Once a straight bone cut 212 has been created across the 1^{st} metatarsal bone 104, as shown in FIG. 8B, the metatarsal head 108 may be pushed in a lateral direction 216 by way of a suitable tool, such as a lever device 220. As will be appreciated, pushing the metatarsal head 108 in the lateral direction 216 causes a distal bone surface 112 of the metatarsal head 108 to move relatively to a proximal bone surface 116 of the 1^{st} metatarsal bone 104. To perform a lateral head shift osteotomy of the 1^{st} metatarsal bone 104, the lever device 220 may be used to push the metatarsal head 108 until the buttress 136 of the bone fusion plate 120 is adjacent to the proximal bone surface 116, as shown in FIG. 8C. With the buttress 136 abutting the proximal bone surface 116, proximal bone screws 128 (see FIGS. 1-2) may be used to fixate the bone fusion plate 120 to the 1^{st} metatarsal bone 104, as described herein.

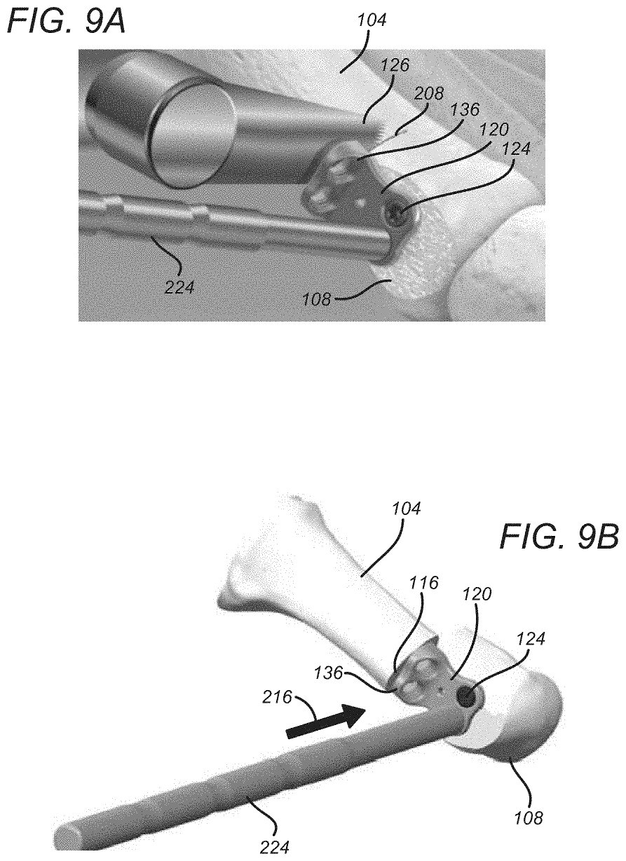

In some embodiments, tools other than the lever device 220 may be used in combination with the bone fusion plate 120 to perform a lateral head shift osteotomy of the 1^{st} metatarsal bone 104. For example, in some embodiments, a threaded handle device 224 may be coupled with the bone fusion plate 120, as shown in FIG. 9A. More specifically, the threaded handle device 224 may be threaded into a distal screw hole 156 and then used to stabilize the bone fusion plate 120 during cutting a straight bone cutline 208 across the 1^{st} metatarsal bone 104. Further, as described herein, the buttress 136 can be used to guide a cutting blade 126 during cutting the 1^{st} metatarsal bone 104. Once the 1^{st} metatarsal bone 104 has been suitably cut, as shown in FIG. 9B, the threaded handle device 224 may be used to push the metatarsal head 108 in the lateral direction 216 until the buttress 136 abuts the proximal bone surface 116. Proximal bone screws 128 (see FIGS. 1-2) may then be used to fixate the bone fusion plate 120 to the 1^{st} metatarsal bone 104, as described herein.

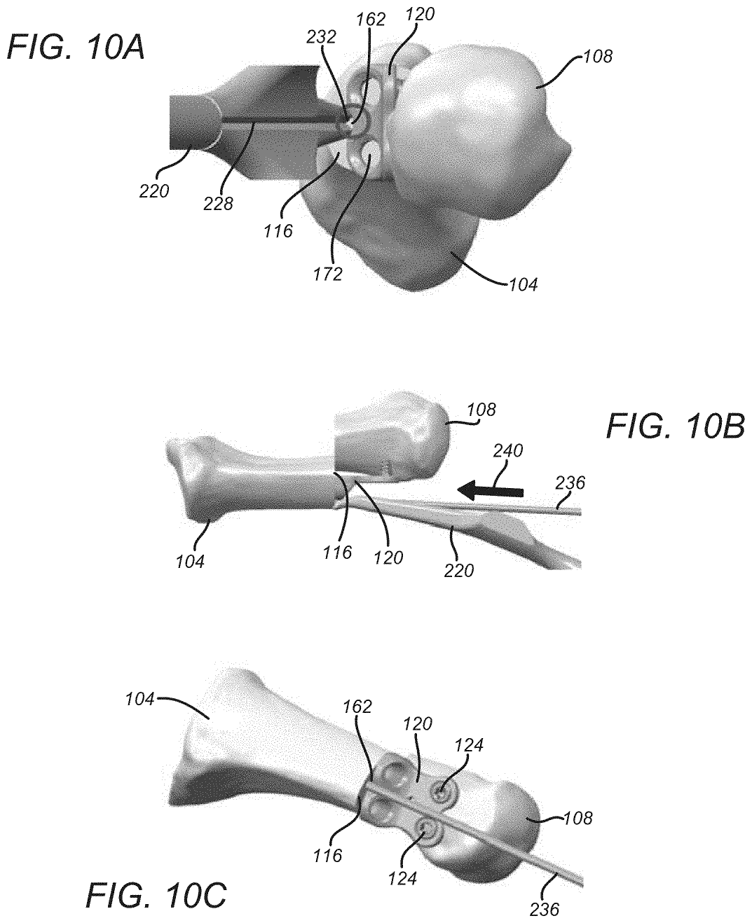

FIGS. 10A through 10C illustrate steps for provisionally fixating the bone fusion plate 120 to the 1^{st} metatarsal bone 104 during preparation of proximal bone holes to receive proximal bone screws 128 (see, for example, FIGS. 1-2). In an exemplary-use environment shown in FIG. 10A, the lever device 220 has been used to move the metatarsal head 108 laterally and thus to position the bone fusion plate 120 adjacent to a proximal bone surface 116 of the 1^{st} metatarsal bone 104. As shown in FIG. 10A, the lever device 220 includes a groove 228 that cooperates with the channel 162 of the bone fusion plate 120 to form an opening 232. As shown in FIG. 10B, a K-wire 236 may be passed through the opening 232 and advanced in a proximal direction 240 through the proximal bone surface 116 and into the 1^{st} metatarsal bone 104 to provisionally fixate the bone fusion plate 120. Once the lever device 220 is removed, as shown in FIG. 10C, the K-wire 236 remains seated in the channel 162, keeping the bone fusion plate 120 advantageously positioned while the proximal bone holes are formed in the 1^{st} metatarsal bone 104. A bone drill and drill guide towers (not shown) may be used to prepare bone holes suitable to receive the proximal bone screws 128. Once the proximal bone screws 128 are inserted into the proximal screw holes 172 and driven into the proximal bone holes in the 1^{st} metatarsal bone, the K-wire 236 may be removed to complete the lateral head shift osteotomy, as shown in FIGS. 1-2.

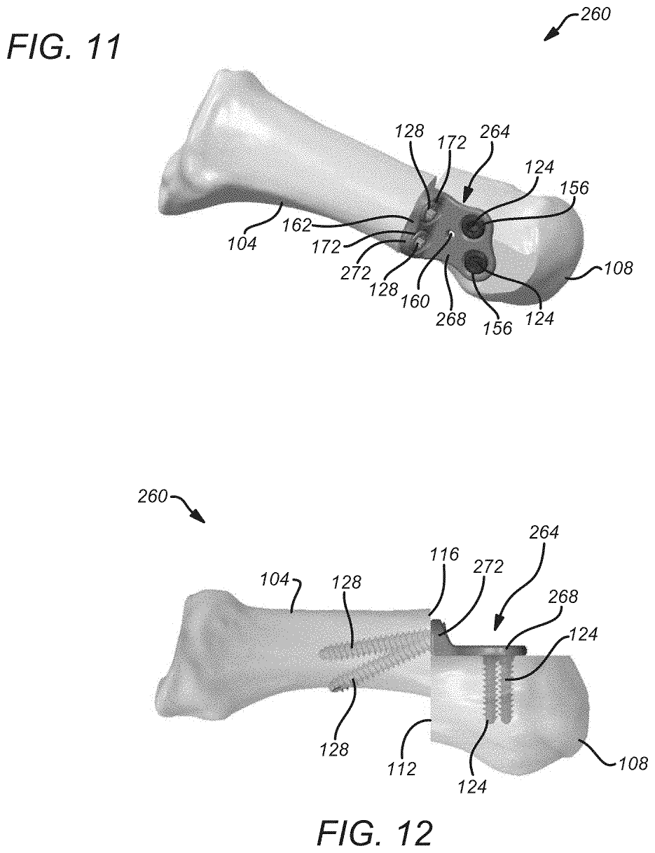

FIGS. 11-12 illustrate an exemplary-use environment wherein a straight distal osteotomy 260 has been performed on a 1^{st} metatarsal bone 104 to correct a hallux valgus deformity of a patient. The straight distal osteotomy 260 is substantially similar to the straight distal osteotomy 100 shown in FIGS. 1-2, with the exception that the straight distal osteotomy 260 includes using an offset bone fusion plate 264 in lieu of the bone fusion plate 120 shown in FIGS. 1-2. As best shown in FIG. 12, a straight bone cut has been performed to separate a metatarsal head 108 from the 1^{st} metatarsal bone 104, and the metatarsal head 108 has been moved in a lateral direction to a suitable position relative to the 1^{st} metatarsal bone 104. The offset bone fusion plate 264 has been attached to the metatarsal head 108 by way of distal bone screws 124 that are driven into bone holes drilled into the side of the metatarsal head 108. Further, the offset bone fusion plate 264 has been attached to the 1^{st} metatarsal bone 104 by way of proximal bone screws 128 that are driven into bone holes drilled into the proximal bone surface 116. As will be appreciated, the 1^{st} metatarsal bone 104 and the metatarsal head 108 will fuse together over time as bone tissue grows across the bone cut.

Upon comparing FIG. 1 and FIG. 11, it will be apparent that the offset bone fusion plate 264 shown in FIG. 11 is substantially similar to the bone fusion plate of FIG. 1. As shown in FIG. 11, the offset bone fusion plate 264 includes an offset plate portion 268 and a buttress 272. The offset plate portion 268 generally is a planar member having a lower, distal surface 140 (see FIG. 4) that is adapted to be attached to the surface of the metatarsal head 108. The buttress 272 is a generally thicker portion of the offset bone fusion plate 264 and includes a proximal surface 144 (see FIGS. 3-4) configured to be attached to the proximal bone surface 116 of the 1^{st} metatarsal bone 104. The distal and proximal surfaces 140, 144 share an intervening buttress angle 152 (see FIG. 7) suitable for fixating the 1^{st} metatarsal bone 104 and the metatarsal head 108 as shown in FIGS. 11-12. The buttress angle 152 may be used to guide cutting the 1^{st} metatarsal bone 104, as described herein. In the embodiment of the offset bone fusion plate 264 shown in FIGS. 11-12, the buttress angle 152 is about 90 degrees. In other embodiments, however, the buttress angle 152 may be greater than 90 degrees, without limitation, as described herein.

As shown in FIG. 11, the offset plate portion 268 includes distal screw holes 156 and a pin hole 160. The distal screw holes 156 are configured to receive distal bone screws 124 for attaching the offset bone fusion plate 264 to the metatarsal head 108. The distal screw holes 156 may include threaded countersinks 164, as described with respect to FIG. 3. The pin hole 160 is configured to enable a practitioner, such as a surgeon, to provisionally position the offset bone fusion plate 264 prior to attaching the offset plate portion 268 to the metatarsal head 108 by way of the distal bone screws 124. For example, a K-wire or an olive wire may be inserted through the pin hole 160 for provisionally attaching the offset plate portion 268 to the metatarsal bone 108. Further, a channel 162 disposed in the buttress 272 may be used by the surgeon to provisionally position the offset bone fusion plate 264 prior to attaching the buttress 272 to the 1^{st} metatarsal bone 104 by way of the proximal bone screws 128, as described herein.

As will be appreciated by those skilled in the art, a primary difference between the bone fusion plate 120 shown in FIG. 1 and the offset bone fusion plate 264 of FIG. 11 is that the offset bone fusion plate 264 includes distal screw holes 156 that are offset from one another. More specifically, the distal screw holes 156 comprising the offset bone fusion plate 264 are disposed in the offset plate portion 268 at different distances from the buttress 272. As shown in FIG. 12, the offset distal screw holes 156 enable the distal bone screws 124 to be positioned at different distances from the buttress 272. As such, the offset distal screw holes 156 enable the offset bone fusion plate 264 to be implemented in a right-foot configuration or a left-foot configuration, as desired. Further, the offset distal screw holes 156 enable the distal bone screws 124 to be positioned farther apart than the distal screw holes 156 comprising the bone fusion plate 120. It is contemplated that the offset distal screw holes 156 provide an improved distribution of the contact force between the offset plate portion 268 and the metatarsal bone 108.

As shown in FIG. 11, the buttress 272 includes proximal screw holes 172 adapted to receive proximal bone screws 128 for attaching the offset bone fusion plate 264 to the 1^{st} metatarsal bone 104. As discussed in connection with FIG. 6, the proximal screw holes 172 preferably include threaded countersinks 176 for threadably engaging a head portion of each proximal bone screw 128 so as to cause the head portion to reside below an exterior surface of the buttress 272 to avoid irritating surrounding soft tissues. Further, the proximal screw holes 172 are disposed in the buttress 272 at a proximal screw angle 180 (see FIG. 5) with respect to the offset plate portion 268. The proximal screw angle 180 preferably is about 25 degrees so as to direct the proximal bones screws 128 into the lateral cortex of the 1^{st} metatarsal bone 104 for fixating the buttress 272 to the 1^{st} metatarsal bone 104. Furthermore, each of the proximal screw holes 172 preferably includes a conical relief 184 (see FIG. 6) that is disposed on a proximal backside of the buttress 272. The conical relief 184 includes an overall angle 188, shown in FIG. 5, comprising about 30 degrees to allow for variable screw angulation as shown in FIG. 12. It is contemplated that the conical relief 184 enables the offset bone fusion plate 264 to be implemented in a right-foot configuration or a left-foot configuration, as desired.

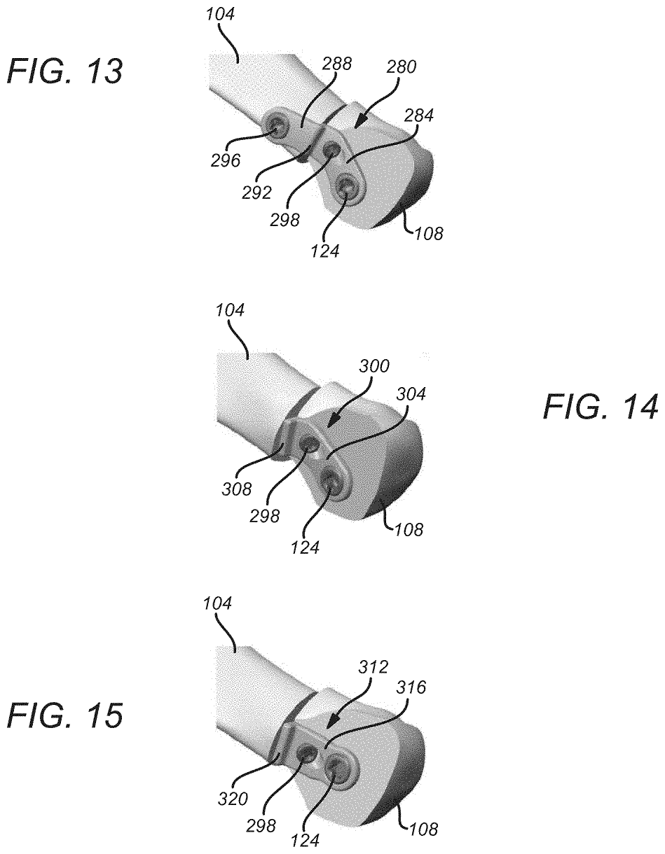

FIG. 13 illustrates an exemplary-use environment wherein a 1^{st} metatarsal bone 104 has been treated with a straight distal osteotomy and an angled bone fusion plate 280 to correct a hallux valgus deformity in accordance with the present disclosure. The angled bone fusion plate 280 includes an angled plate portion 284 and a proximal plate portion 288 that share an intervening buttress 292. In the exemplary-use environment shown in FIG. 13, a straight bone cut has been performed to separate a metatarsal head 108 from the 1^{st} metatarsal bone 104, and the metatarsal head 108 has been moved in a lateral direction to a suitable position relative to the 1^{st} metatarsal bone 104.

The angled plate portion 284 has been attached to the metatarsal head 108 by way of a distal bone screw 124 driven into a bone hole drilled into the metatarsal head 108. The proximal plate portion 288 has been attached to the 1^{st} metatarsal bone 104 by way of a proximal straight bone screw 296 driven into a bone hole drilled into the 1^{st} metatarsal bone 104. Further, a proximal angled screw 298 driven across the bone cut and into the 1^{st} metatarsal bone 104 has been used to attach the buttress 292 between the 1^{st} metatarsal bone 104 and the metatarsal head 108. It is contemplated that the 1^{st} metatarsal bone 104 and the metatarsal head 108 will fuse together over time as bone tissue grows across the bone cut.

FIG. 14 illustrates an exemplary-use environment wherein a 1^{st} metatarsal bone 104 has been treated with a straight bone cut and an angled bone fusion plate 300 to correct a hallux valgus deformity in accordance with the present disclosure. The angled bone fusion plate 300 is substantially similar to the angled bone fusion plate 280 shown in FIG. 13, with the exception that the angled bone fusion plate 300 lacks the proximal plate portion 288 comprising the angled bone fusion plate 280. As such, the angled bone fusion plate 300 includes an angled plate portion 304 and a buttress 308.

As shown in FIG. 14, the straight bone cut has been performed to separate a metatarsal head 108 from the 1^{st} metatarsal bone 104, and the metatarsal head 108 has been moved in a lateral direction to a suitable position relative to the 1^{st} metatarsal bone 104. The angled plate portion 304 has been attached to the metatarsal head 108 by way of a distal bone screw 124 driven into a bone hole drilled into the metatarsal head 108. The buttress 308 has been attached to the 1^{st} metatarsal bone 104 by way of a proximal angled screw 298 driven into a bone hole drilled across the bone cut, from the metatarsal head 108 and into the 1^{st} metatarsal bone 104. As described herein, the 1^{st} metatarsal bone 104 and the metatarsal head 108 will fuse together over time as bone tissue grows across the bone cut.

FIG. 15 illustrates an exemplary-use environment wherein a 1^{st} metatarsal bone has been treated with a straight bone cut and a straight bone fusion plate 312 to correct a hallux valgus deformity in accordance with the present disclosure. As will be appreciated, the straight bone fusion plate 312 is substantially similar to the angled bone fusion plate 300 shown in FIG. 14, with the exception that the straight bone fusion plate 312 includes a straight plate portion 316 in lieu of the angled plate portion 304 comprising the angled bone fusion plate 300 of FIG. 14. Thus, the straight bone fusion plate 312 includes the straight plate portion 316 and a buttress 320.

In the exemplary-use environment of FIG. 15, the straight bone cut has been performed to separate the metatarsal head 108 from the 1^{st} metatarsal bone 104, and the metatarsal head 108 has been moved in a lateral direction to a suitable position relative to the 1^{st} metatarsal bone 104. The straight plate portion 316 has been attached to the metatarsal head 108 by way of a distal bone screw 124 driven into a bone hole drilled into the metatarsal head 108. The buttress 320 has been attached to the 1^{st} metatarsal bone 104 by way of a proximal angled screw 298 driven into a bone hole drilled into the metatarsal head 108, across the bone cut, and into the 1^{st} metatarsal bone 104. As described herein, the 1^{st} metatarsal bone 104 and the metatarsal head 108 will fuse together over time as bone tissue grows across the bone cut.

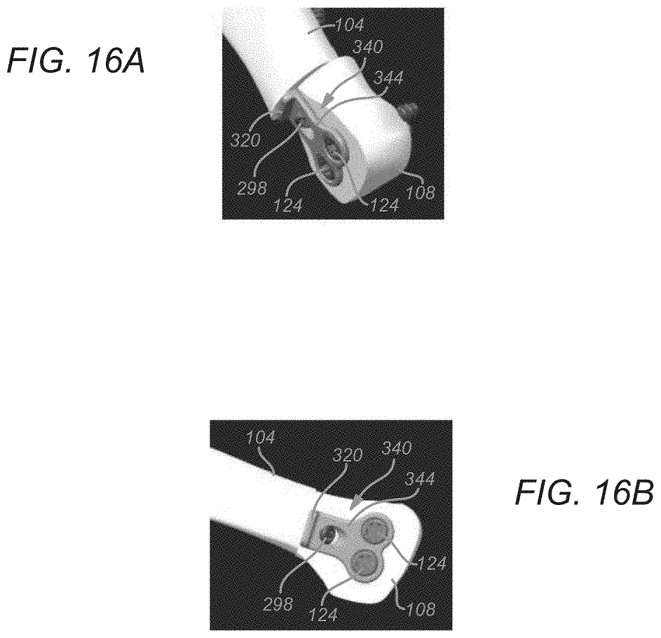

FIGS. 16A-16B illustrate an exemplary-use environment wherein a 1^{st} metatarsal bone has been treated with a T-shaped bone fusion plate 340 to correct a hallux valgus deformity in accordance with the present disclosure. The T-shaped bone fusion plate 340 is substantially similar to the straight bone fusion plate 312 shown in FIG. 15, with the exception that the T-shaped bone fusion plate 340 of FIGS. 16A-16B includes a T-shaped plate portion 344 in lieu of the straight plate portion 316 comprising the straight bone fusion plate 312 of FIG. 15. As such, the T-shaped bone fusion plate 340 includes the T-shaped plate portion 344 and a buttress 320.

In the exemplary-use environment of FIGS. 16A-16B, the straight bone cut has been performed to separate the metatarsal head 108 from the 1^{st} metatarsal bone 104, and the metatarsal head 108 has been moved in a lateral direction to a suitable position relative to the 1^{st} metatarsal bone 104. The T-shaped plate portion 344 has been attached to the metatarsal head 108 by way of a pair of distal bone screws 124 driven into bone holes drilled into the metatarsal head 108. In the embodiment illustrated in FIGS. 16A-16B, the T-shaped plate portion 344 positions the distal bone screws 124 in a side-by-side arrangement. In some embodiments, however, the T-shaped plate portion 344 may be configured to position the distal bone screws 124 in arrangements other than side-by-side, such as, for example, an offset disposition, as described herein with respect to FIGS. 11-12, without limitation.

With continuing reference to FIGS. 16A-16B, the buttress 344 has been attached to the 1^{st} metatarsal bone 104 by way of a single proximal angled screw 298 driven into a bone hole drilled into the metatarsal head 108, across the bone cut, and into the 1^{st} metatarsal bone 104. It is contemplated that the T-shaped bone fusion plate 340 fixates the bone cut such that the 1^{st} metatarsal bone 104 and the metatarsal head 108 will fuse together over time as bone tissue grows across the bone cut.

While the bone fusion plate, screw constructs, and methods have been described in terms of particular variations and illustrative figures, those of ordinary skill in the art will recognize that the bone fusion plate and screw constructs are not limited to the variations or figures described. In addition, where methods and steps described above indicate certain events occurring in certain order, those of ordinary skill in the art will recognize that the ordering of certain steps may be modified and that such modifications are in accordance with the variations of the bone fusion plate and screw constructs. Additionally, certain of the steps may be performed concurrently in a parallel process, when possible, as well as performed sequentially as described above. To the extent there are variations of the bone fusion plate and screw constructs, which are within the spirit of the disclosure or equivalent to the bone fusion plate and screw constructs found in the claims, it is the intent that this patent will cover those variations as well. Therefore, the present disclosure is to be understood as not limited by the specific embodiments described herein, but only by scope of the appended claims.

What is claimed is:

1. A bone fusion plate for treating hallux valgus, comprising:
a plate portion comprising a lower, distal surface; and
a buttress comprising a proximal surface.

2. The bone fusion plate of claim 1, wherein the buttress includes one or more proximal screw holes configured to receive bone screws for attaching the buttress to a proximal bone surface of a metatarsal bone.

3. The bone fusion plate of claim 2, wherein any one or more of the one or more proximal screw holes includes a conical relief configured to allow for variable screw angulation.

4. The bone fusion plate of claim 2, wherein any one or more of the one or more proximal screw holes includes a threaded countersink for threadably engaging a head portion of a proximal bone screw.

5. The bone fusion plate of claim 2, wherein the one or more proximal screw holes are disposed in the buttress at a proximal screw angle with respect to the plate portion.

6. The bone fusion plate of claim 2, wherein any one or more of the one or more proximal screw holes includes a conical relief adapted to allow for variable screw angulation for a range of osteotomy shifts.

7. The bone fusion plate of claim 1, wherein the plate portion includes one or more distal screw holes configured to receive bone screws for attaching the plate portion to a metatarsal head.

8. The bone fusion plate of claim 7, wherein the one or more distal screw holes are positioned at different distances from the buttress.

9. The bone fusion plate of claim 7, wherein any one or more of the one or more distal screw holes includes a threaded countersink for threadably engaging a head portion of a distal bone screw.

10. The bone fusion plate of claim 1, wherein the proximal surface is configured to be attached to a proximal bone surface formed by cutting a metatarsal bone and laterally shifting the metatarsal head.

11. The bone fusion plate of claim 10, wherein the proximal surface and the distal surface share an intervening buttress angle suitable for fixating the metatarsal bone and the metatarsal head.

12. The bone fusion plate of claim 10, wherein the buttress angle comprises a cut guide configured to guide cutting the metatarsal bone.

13. The bone fusion plate of claim 10, wherein the buttress angle ranges between about 70 degrees and about 110 degrees.

14. A lateral shift osteotomy buttress plate, comprising:
a plate portion for attaching to a distal bone portion; and
a buttress for attaching to a proximal bone portion.

15. The buttress plate of claim 14, wherein the buttress includes a proximal surface disposed at a buttress angle with respect to a distal surface comprising the plate portion.

16. The buttress plate of claim 15, wherein the proximal surface comprises a cut guide for performing a bone cut to separate the distal bone portion and the proximal bone portion.

17. The buttress plate of claim 16, wherein the buttress angle ranges between about 70 degrees and about 110 degrees.

18. The buttress plate of claim 14, wherein the buttress includes one or more proximal screw holes disposed at a proximal screw angle with respect to the plate portion and configured to receive bone screws for attaching the buttress to the proximal bone portion.

19. The buttress plate of claim 18, wherein any one or more of the one or more proximal screw holes includes a threaded countersink for threadably engaging a head portion of a proximal bone screw.

20. The buttress plate of claim 18, wherein any one or more of the one or more proximal screw holes includes a conical relief configured to allow for variable screw angulation for a range of osteotomy shifts.
